(19) United States
(12) Patent Application Publication
(10) Pub. No.: US 2012/0330209 A1
(43) Pub. Date: Dec. 27, 2012

(54) Non-leaching Antimicrobial Wound Dressing

(76) Inventors: Zhiqiang Song, Newtown, CT (US); Ted Deisenroth, Brookfield, CT (US)

(21) Appl. No.: 13/528,289

(22) Filed: Jun. 20, 2012

Related U.S. Application Data
(60) Provisional application No. 61/599,601, filed on Feb. 16, 2012.
(60) Provisional application No. 61/603,564, filed on Feb. 27, 2012.
(60) Provisional application No. 61/500,385, filed on Jun. 23, 2011.

(51) Int. Cl. A61F 13/00; B05D 7/00

(52) U.S. Cl. 602/48; 602/52; 427/2.31

(57) ABSTRACT

The present application discloses preparations of antimicrobial wound dressings. Strongly binding polymeric dialkyl aminoalkyl(meth)acrylates are used to treat highly absorbent wound dressing materials. The resulting finish is highly antimicrobial effective and non-leachable.

This application takes the benefit of U.S. Provisional Application Nos. 61/500,385 filed on Jun. 23, 2011, 61/599,601 filed Feb. 16, 2012 and 61/603,564 filed Feb. 27, 20012 the contents of which are herein incorporated entirely by reference.

### FIELD OF THE INVENTION

The present invention relates to antimicrobial wound dressing materials and methods for their preparation. The materials are made antimicrobial by incorporation of an antimicrobial dialkyl aminoalkyl(meth)acrylate polymer into a coating or finish on the wound dressing material. The wound dressing materials are typically a gauze, pad, band aids, absorptive packings, cotton or cotton balls, wound fillers and tapes which may be fibrous, woven or nonwoven, solid, or flexible mass of material upon which the antimicrobial polymers can be applied and suitable for use on a wound. The antimicrobial polymers are non-leachable once applied to the wound dressing.

### BACKGROUND

Wound healing is a complicated process. Infections adversely impact wound repair through ongoing chronic inflammation and production of toxic molecules and metabolites from both the microbe and the immune response. Skin wounds harbor microbes that can infect the compromised tissue. Thus microbial proliferation must be controlled or prevented to enable proper healing.

Moreover, the biofouling of bodily fluids is a serious problem for wound dressings because the decomposition of bodily fluids in a wound dressing can lead to proliferation of infections and malodor. Thus, there is a need for the materials of the contact layer and the absorbent layer of a wound dressing to have antimicrobial properties that prevent or at least limit the ability of bacteria to grow and spread inside the wound dressing.

In designing a material for dressing the wound, in particular one that is capable of providing adequate antimicrobial activity, it is desired that the antimicrobial substance(s) present in the dressing do not compromise the physiologic healing, and the repair-promoting physiochemical aspects of the wound matrix itself. To be beneficial, the antimicrobial agent preferably exerts its effect over the relevant time scale of days, without being washed out by tissue fluid flows or somehow altered by exposure to tissue fluids.

One approach to designing antimicrobial wound dressings is to modify the surface of the medical fabric or dressing by treatment with antimicrobial agents.

However, there are certain requirements of such fabrics or dressings. Firstly, the medical fabric or dressing needs to retain its activity over time. For applications in which the antimicrobial modified material will come into contact with aqueous biological fluids, it is important that the antimicrobial agent is not rinsed away, or otherwise inactivated.

It is also important that the agent responsible for the antimicrobial effect does not leach into the wound as this might result in compromising healing of the wound or cause other undesired consequences (e.g. leaching into open wounds, leaching onto products intended for human consumption, and staining of skin).

The use of particular polymers as antimicrobial agents is known in the art. It is further known that antimicrobial polymers may be used to treat surfaces to make surfaces antimicrobial and be incorporated into other polymers by blending and still retain microbiocidal activity.

In particular, there are a number of references which recognize the antimicrobial activity of polymeric dialkyl aminoalkyl(meth)acrylate polymers.

For example, G. J. Gabriel et al., Materials Science and Engineering R 57 (2007), page 28-64 and C. J. Hewitt et al., Biotechnology Letters 26: pages 549-557, 2004, Co-pending U.S. Provisional Ser. No. 61/500,385, filed on Jun. 23, 2011, European Application No. 0204312, U.S. Pat. Nos. 5,967,714, 6,203,856, 6,096,800, 6,316,044, 6,790,910, WO2002058757, US 20080193497 and PCT application WO200217725 all teach variations of antimicrobial polymers.

The above references have certain disadvantages. Grafting of aminoalkylmethacrylate monomer or polymers to surfaces requires an additional grafting step and may adversely impact the physical properties of the grafted material or the ungrafted versions leach.

The disadvantages cited above are magnified when the application is an antimicrobial effective wound dressing or medical materials that are likely to come in contact with biological fluids and open wounds/sores. Such materials should be relatively inexpensive and accordingly any grafting or covalent linkage step to permanently tie the antimicrobial to a surface would be a serious disadvantage. Further the grafting or covalent linking is likely to impact the physical properties of the dressing such as absorbency, feel and texture properties.

Additionally, the wound dressing should not adhere to newly formed and delicate tissue (formed as a result of the healing process). Accordingly, the antimicrobial treatment should not impact the “sticking” of the dressing to this newly formed tissue as this would disrupt it's integrity and thus the healing process. Any antimicrobial finish on the dressing should adhere strongly to the dressing but not increase the adherence of the dressing to the healing wound surface.

It would further be advantageous if the antimicrobial dressing finish could be made to be effective at lower active loadings, effective against fungi, adhere strongly to dressing substrates without additional use of binders, not leach into the wound and still maintain antimicrobial activity over time.

### SUMMARY OF THE INVENTION

The present inventors have devised a solution to many of the above problems. Specifically the inventors have discovered that when particular amine containing (meth)acrylates are used to treat wound dressing materials, the amine containing (meth)acrylates are non-leachable even after exposure to water/biological fluids and thus will not migrate into the wound or open sore. Further, the antimicrobial polymers maintain their microbial effect over the time of use even after exposure to fluids and are capable of binding well to the dressing without the use of additional binders and without grafting or covalent linking the antimicrobial finish to the dressing.

Accordingly, this application is directed to several embodiments:

An antimicrobial, non-leachable wound dressing,
A method of preparing the same,
and
Use of the antimicrobial polymer to form a non-leachable antimicrobial wound dressing.

Thus one embodiment includes an antimicrobial, non-leachable wound dressing which comprises a wound-dressing, especially a gauze, treated with a polymer formed from the monomer of formula (I)

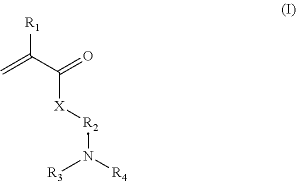

wherein R₁ is H or CH₃,
R₂ is C₁-C₅ alkyl bi-radical, preferably R₂ is C₂ bi-radical,
R₃ and R₄ are independently H or C₁-C₅ alkyl radical which can be linear or branched,
preferably R₃ is hydrogen and R₄ is tert-butyl,
and X is a divalent radical of —O—, —NH— or —NR₅, preferably —NH, wherein R₅ is C₁-C₆alkyl.
The non-leachability is determined by ASTM E 2149 method using the specified pre-test and post-test designed for testing non-leachable antimicrobial surfaces.

Furthermore, the application is directed to a method of forming an antimicrobial non-leachable wound dressing comprising the steps

a) treating a wound dressing and/or wound filler with a polymer formed from a monomer of formula (I)

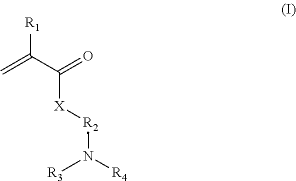

wherein R₁ is H or CH₃,
R₂ is C₁-C₅ alkyl bi-radical, preferably R₂ is C₂ alkyl bi-radical,
R₃ and R₄ are independently H or C₁-C₅ alkyl radical, which can be linear or branched,
preferably R₃ is hydrogen and R₄ is tert-butyl, and X is a divalent radical of —O—, —NH— or —NR₅, preferably —NH, wherein R₅ is C₁-C₆alkyl,
wherein the formed polymer is optionally dissolved or dispersed in a liquid.
The non-leachability may be determined by ASTM E 2149 method using the specified pre-test and post-test designed for testing non-leachable antimicrobial surfaces.

Use of a polymer formed from the monomer of formula (I) to form a non-leachable antimicrobial wound dressing.

### DETAILED DESCRIPTION OF THE INVENTION

### Definitions

“Comprising” for purposes of the invention is open ended, that is other components may be included. Comprising is synonymous with containing or including.

When the term “molecular weight” is used this will normally indicate weight average molecular weight (Mw) unless otherwise indicated.

“Wound dressing” materials for purposes of this application means materials which are suitable for direct exposure to wounds of any kind including but not limited to wounds such as burns, pressure sores, punctures, ulcers, abrasions, cuts, incisions, sores including bed sores and suture treated areas on skin.

The term “wound dressing” includes the term “wound fillers” as “wound fillers” are products that are typically used for the treatment of penetrating wounds to the skin and would include such materials as foam dressings, gels, network polymers, or absorbent polymeric matrices which may be applied over large penetrating wounds to the skin and underlying tissues. The fillers are frequently flexible and compressibly conform with areas proximal to the wound. Wound fillers present particular problems because bacteria will grow in exudate that is absorbed by the filler. Bacteria grow in the exudate and create unpleasant odors as well as a risk of infecting the wound site if the dressing is not timely changed.

Furthermore, absorptive packings are also a likely material to benefit from treatment with the antimicrobial polymer. For example, there are a number of instances where absorptive packings are placed in natural body orifices with significant possibility for dangerous bacterial growth. Various nasal packings can become bacterially laden following insertion into nasal passageways. Numerous deaths have resulted from “toxic shock syndrome” resulting from multiplication of Staphylococcus aureus bacteria in feminine care products, particularly tampons. There have been a large number of related problems. For example, U.S. Pat. No. 5,641,503, to Brown-Skrobot, seeks to produce a germicidal tampon and contains a useful list of references to the toxic shock problem. Use of the antimicrobial polymer on such absorptive packings is also envisioned for the purpose of controlling bacterial proliferation.

Thus “wound dressing” would include materials in the form of a gauze, pads, band aids, wound fillers, absorptive packings, pads, cotton or cotton balls, sponges or tapes, especially a gauze.

The wound dressing materials or substrates are selected from the group consisting of synthetic materials, blends of synthetic materials and blends of synthetic materials with cotton, blends of synthetic materials with cellulose or cellulose derivatives, silk, cotton and cellulosics.

Preferably, the wound dressing materials or substrates are at least partially derived from synthetic materials. Most preferably, the wound dressing material is in the form of a gauze, fibers, nonwovens, pads, band aids, wound fillers, absorptive packings, sponge or tapes, especially a gauze.

Synthetic materials for purposes of this application means at least partially synthetically derived polymeric materials in the form of fibers, nonwovens, sponges, absorptive packings, pads, cotton or cotton balls, band aids, sponges or tapes, especially a gauze. For example the synthetically derived polymeric materials may be selected from the group consisting of rayon, polyester, polyethylene, polypropylene, cellulose, cellulose derivative and their blends.

Preferably, the dressing materials will typically comprise synthetic polymeric fibers or nonwovens formed at least partially from rayon, polyester, polyethylene, polypropylene and blends thereof with cellulose or cellulose derivative.

The gauze, pad, band aids, absorptive packings, cotton or cotton balls, wound fillers, sponges and tapes may be fibrous, woven or nonwoven, solid, or flexible mass of material upon which the antimicrobial polymers can be applied but suitable for use on a wound.

A highly preferred gauze or open mesh fibrous network includes several materials manufactured by Johnson & Johnson Company (J&J). J&J's, NU GAUZE®, general use sponge, J&J's “STERILE GAUZE® Mirasorb sponge”, J&J's “SOFT WICK®” dressing sponge and hospital grade gauze pads by Johnson & Johnson. These materials are for example, rayon/cellulose/polyester sheets with non-woven mesh-like structures, and a fiber surface area greater than traditional woven cotton-fiber gauze.

For purposes of this application antimicrobial effect means the polymer is effective against pathogenic gram negative, gram positive bacteria, yeast, fungal and/or mold organisms and against bacteria of the skin flora.

(Meth)acrylate means methacrylate or acrylate and likewise (meth)acrylamide means methacrylamide or acrylamide.

By “non-leaching” is meant that the antimicrobial polymer, once attached to the wound dressing material, do not appreciably separate from, migrate out of, or away from the material or substrate, enter the wound, or otherwise become non-integral with the material or substrate under normal uses. Non-leaching antimicrobial surfaces, for example, can be determined by ASTM E2149 method with the pre-test and post-test methods. ASTM E2149 is specifically designed to measure antimicrobial non-leaching. The ASTM E2149 makes the non-leaching determination by the absence of a zone of inhibition.

The antimicrobial finish or treatment to the wound dressing material is as explained above non-leaching. However, the antimicrobial polymer does not need to be chemically attached (covalently bonded) to the wound dressing material. This is one of the clear benefits of the particular antimicrobial polymer. The application of the antimicrobial polymer in a solution or dispersion directly to the wound dressing material via a simple dipping, spraying or coating step will insure sufficient binding of the antimicrobial polymer to the dressing material to prevent leaching even under exposure to fluids.

When the term “non-leachable” is used in accordance with this application, the modifier “non-leachable’ refers to the antimicrobial polymer finish per se. It is possible that the wound dressing may contain in addition to the antimicrobial polymer formed from formula (I), other ingredients which are entirely meant to leach from the wound dressing.

Thus the antimicrobial wound dressing may comprises a wound-dressing material, treated with a polymer formed from the monomer of formula (I)

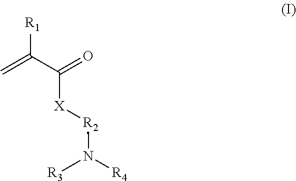

wherein R₁ is H or CH₃,
R₂ is C₁-C₅ alkyl bi-radical, preferably R₂ is C₂ bi-radical,
R₃ and R₄ are independently H or C₁-C₅ alkyl radical which can be linear or branched,
preferably R₃ is hydrogen and R₄ is tert-butyl,
and X is a divalent radical of —O—, —NH— or —NR₅, preferably —NH, wherein R₅ is C₁-C₆ alkyl, and
may further contain other medicinal or antimicrobial ingredient which may be leach such as antibiotics or antimicrobials such as silver, silver salts, quaternary ammonium salts polyhexamethylen-biguanid compounds, polydimethyldiallyammonium chloride, chlrohexidine, and in particular phenoxyethanol. 5-Chloro-2-(2,4-dichlorophenoxy)-phenol, which is marketed by BASF SE Ludwigshaven, Germany under the name of Irgasan®

The non-leachability may be determined by ASTM E 2149 method using the specified pre-test and post-test designed for testing non-leachable antimicrobial surfaces.

Thus the antimicrobial polymer while non-leaching is preferably essentially not covalently bound to the wound dressing substrate or material.

The antimicrobial polymer is for example, pre-formed before applied to the wound dressing material. Accordingly the monomer of formula (I) is not polymerized in the presence of the wound dressing material or is not covalently grafted to the wound dressing material.

The antimicrobial polymer on the wound dressing may be protonated, partially protonated or not protonated.

The loading on the wound dressing for purposes of this application means the weight percent of the polymer formed from formula (I) on the dressing is based on the weight of the total dressing after drying, that is after solvents have evaporated. As a dressing typically is characterized by a high absorbent surface area, a 1 or 3% solution of the dispersed/dissolved polymer will end up forming a loading onto the dressing after drying that may range from 2, 3 to 4 times the solution concentration. For example, when a 1 wt. % solution of the polymer formed from formula (I) is used to treat the dressing, the finished dressing may result in an antimicrobial loading to the dressing which may contain the antimicrobial polymer ranging from 2, 3, 4, 5 or 6 wt. % of the total weight of the dressing. The loading will depend on a number of factors such as the absorptive capacity of the dressing, the area of the dressing treated, the concentration of the solution with polymer used to treat the dressing and the antimicrobial polymer per se.

The Antimicrobial Polymer

The antimicrobial polymer may be water soluble or water insoluble. Preferably, however, the antimicrobial polymer on the wound dressing is substantially water-insoluble.

Water-insoluble for purposes of this application means<5%, preferably <1% soluble in deionized water at room temperature (25° C.) and pressure. The term substantially “water-insoluble” for purposes of this application means that less than 5 wt. %, preferably less than 3 wt. %, most preferably less than 1 wt. % and especially 0.5 or 0.1 wt. %, most especially <100 ppm or <10 ppm of the antimicrobial polymer is soluble in deionized water at room temperature (25° C.) and pressure. For example, the antimicrobial oligomer according to formula (II) may be <10 ppm soluble in deionized water at room temperature.

The antimicrobial polymers are made by polymerizing an alkylaminoalkyl(meth)acrylate or an alkylaminoalkyl(meth)acrylamide monomer.

Suitable alkylaminoalkyl(meth)acrylate and alkylaminoalkyl(meth)acrylamide monomers may be represented by general formula (I):

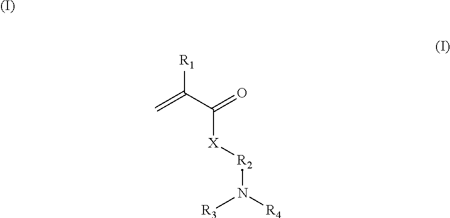

wherein
R₁ is H or CH₃,
R₂ is C₁-C₅alkyl bi-radical,
R₃ and R₄ are independently H or C₁-C₅alkyl radical which can be linear or branched,
and X is a divalent radical of —O—, —NH— or —NR₅, wherein R₅ is C₁-C₆ alkyl.
Preferably, R₂ is a C₂ bi-radical,
R₃ is hydrogen and R₄ is C₄, especially tert-butyl,
and X in —NH.

Preferred monomers from formula (I) are 2-tert-butylaminoethyl(meth)acrylate (tBAEMA), 2-dimethylaminoethyl(meth)acrylate, 2-diethylaminoethyl(meth)acrylate, 3-dimethylaminopropyl(meth)acrylate, N-3-dimethylaminopropyl(meth)acrylamide, and N-3-diethylaminopropyl(meth)acrylamide with the most preferred being 2-tert-butylaminoethyl(meth)acrylate (tBAEMA).

The polymer of formula (I) may be formed from a monomer meeting the description of formula (I) only or may be formed from additional monomers. For example, the polymer may be formed from one or more monomers of formula (I) selected from the group consisting of -tert-butylaminoethyl(meth)acrylate (tBAEMA), 2-dimethylaminoethyl(meth)acrylate, 2-diethylaminoethyl(meth)acrylate, 3-dimethylaminopropyl(meth)acrylate, N-3-dimethylaminopropyl(meth)acrylamide, and N-3-diethylaminopropyl(meth)acrylamide.

Alternatively, the polymer may be formed from the monomers of formula (I) and additional monomers not meeting the definition of formula (I).

The additional monomers not meeting the definition of formula (I) however, should not interfere with the antimicrobial effect of repeating units of formula (I).

Suitable co-monomers not meeting the definition of formula (I) are those which are substantially water-insoluble. Example of suitable co-monomers include, but not limit to, styrene, esters of (meth)acrylic acid such as methyl methacrylate, amides of (meth)acrylic acid such as N,N-dimethylacrylamide and N-isopropylacrylamide, and olefins such as ethylene and propylene.

While the polymer formed from monomers of formula (I) may be a co-polymer, it is believed that there should be significant connected repeat monomers units of monomers of formula (I). For example, if the polymer formed from monomers of formula (I) is a co-polymer, the co-polymer may be a block co-polymer containing at least one block formed from monomer units of formula (I). Alternatively, a suitable random copolymer can be a random copolymer preferably with a monomer of formula (I) content of greater than 50%, 60% or 80%.

For example, a suitable tBAEMA copolymer can be a block copolymer preferably with a tBAEMA content of greater than 10% and a tBAEMA block comprising more than 10 tBAEMA monomeric units.

Alternatively, the co-polymer could have a grafted or a brush architecture wherein the co-polymer contains pendant graft monomer repeat units of formula (I) along a linear polymer chain. Hyperbranched architectures are also envisioned wherein a central multifunctional acrylate may be polymerized with monomers of formula (I) giving a star like or hyperbranched configuration wherein the monomer repeat units of formula (I) radiate around the central multifunctional acrylate.

Accordingly, the antimicrobial polymer may have most any architecture and can be a linear polymer formed from monomer(s) of formula (I), a linear homopolymer of only one type of monomer of formula (I), a graft, brush or comb co-polymer having pendant monomer repeat units of formula (I) or a star like polymer configuration wherein the repeat units of formula (I) radiate outward from a central multifunctional (meth)acrylate.

However, preferably the polymer is formed only from monomers meeting the definition of formula (I). While the antimicrobial polymer may be a co-polymer it is preferably that the polymer is a homopolymer.

It is most preferable that the homopolymer is formed from tBAEMA.

The polymer formed from monomer of formula (I), especially the polymer formed from tBAEMA may or may not be crosslinked.

Suitable non-leachable antimicrobial polymers of formula (I) include homopolymer and copolymers with weight average molecular weights ranging from 500 to 5,000,000 g/mole, preferably from 1,000 to 200,000 g/mole. The preferred antimicrobial polymer is a low weight average molecular weight (MW<20,000) with a narrow MW distribution (polydispersity Mw/Mn<4). Most preferably the antimicrobial polymer has a weight average molecular weight (Mw) ranging from 400 to 20,000 g/mole and especially from 400 to 10,000 or even 1000 to 10,000.

The average molecular weights of polymers formed from formula (I) are measured by gel permeation chromatography (GPC) using poly(methyl methacrylate) narrow molecular weight standards.

The polymers formed from formula (I) preferably have a molecular weight distribution with a polydispersity index (PDI=Mw/Mn) of 1.0 to 4.0 with the weight average molecular ranging from 400 to 20,000 g/mole, preferably the molecular weight distribution ranges from 1.0 to 3.0.

Most preferably, the polymers formed from formula (I) have a weight average molecular weight Mw ranging from 1000 to 10,000 with a PDI ranging from 1.0 to 4.0 or from 1.0 to 3.0.

### Preparation of the Antimicrobial Polymers

The antimicrobial alkylaminoalkyl polymers can be prepared by virtually any conventional random radical polymerization, controlled radical polymerization (CRP), anionic polymerization and cationic polymerization with reaction conditions aimed for virtually any molecular weight polymers known to the art skilled. The preparation can be carried out using various polymerization techniques such as solution, emulsion, microemulsion, inverse emulsion, and/or bulk polymerizations, as well as other technologies that are available to those who are skilled in the art.

Molecular weights of polymers synthesized by radical polymerization, anionic polymerization and cationic polymerization can be controlled by varying reaction conditions such as initiator type and concentration, monomer concentration, reaction temperature, chain transfer agent type and concentration. Generally, high concentration of initiator, low concentration of monomer, high reaction temperature and addition of a chain transfer agent are used to achieve low molecular weights for the antimicrobial polymers.

Conventional random radical polymerization provides a simple way to make the antimicrobial polymers. The source of free radicals required to initiate the polymerization of the radically polymerizable monomers is a free radical initiator. The free radicals may be formed by thermal or photoinduced decomposition of the initiator or by a redox reaction with the initiator.

Typical free radical initiators include, but not limited to, azo and peroxide compounds. Typical azo initiator include azobis(isobutyronitrile) (AIBN), dimethyl 2,2′-azobisisobutyrate (MAIB), 1,1′-azobis(1-cylcohexanenitrile), 2,2′-azobis(2,4,4-trimethylpentane), and azobis-2,4-dimethylvaleronitrile, polymeric or oligomeric materials comprising azo, —N═N—, groups. Water soluble azo initiator may be used in emulsion polymerization and selected from the group consisting of 2,2-azobis-(N,N′-dimethylene-isobutyramidine) dihydrochloride, 2,2′-azobis-(2-amidinopropane)dihydrochloride, 4,4′-azobis-(4-cyanopentane-carboxylic acid); 2,2′-Azobis[2-(5-methyl-2-imidazolin-2-yl)propane]dihydrochloride; 2,2′-Azobis[N-(2-carboxyethyl)-2-methylpropionamidine]tetrahydrate; 2,2′-Azobis[2-(3,4,5,6-tetrahydropyrimidin-2-yl)propane]dihydrochloride; and 2,2′-Azobis{2-methyl-N-[2-(1-hydroxybuthyl)]propionamide.

Typical peroxide radical initiator may include acyl and diacyl peroxides, alkyl peroxides, dialkyl peroxydicarbonates, hydroperoxides such as tert.-butylhydroperoxide, peresters, and inorganic peroxides such as hydrogen peroxide, ammonium persulfate, potassium persulfate and sodium persulfate, benzoyl peroxide (BPO) or a peroxy acid such as peroxyacetic acid or peroxybenzoic acid. The redox initiator in combination with reducing agents is selected from the group consisting of, for example, an acyl peroxides with tertairyamine such as triethylamine, and tert.-butylhydroperoxide or persulfate with iron(II)-ammonium sulfate, ascorbic acid, sodium methyl sulfinate, disodium disulfite, sodium hydrogen sulfite, sodium phosphite, potassium phosphate, hydrogen phosphite, sodium hypophosphite or potassium hypophosphite.

Azo initiator such as AIBN is preferably used at high concentration from 1% to 20% based on monomer to achieve low molecular weight using radical polymerization to prepare the antimicrobial oligomers. Lower concentration of initiator may be used in combination with an effective chain transfer agent to obtain low molecular weight.

Suitable chain transfer agents may include mercaptans such as dodecyl mercaptan, octyl mercaptan, hexyl mercaptan and ethanolmercaptan and halogen-containing compounds such as carbon tetrabromide.

However, controlled living polymerization methods may also be used for preparing the antimicrobial polymers. Living polymerization techniques have been traditionally used for the synthesis of well-defined polymers where polymerization proceeds in the absence of irreversible chain transfer and chain termination, i.e. nearly ideally in anionic polymerization and less ideally in cationic polymerization. Anionic living polymerization is initiated by nucleophilic addition to the double bond of the monomer using an organo-metallic initiator such as an alkyl lithium or Grignard reagent. An alternative means of initiation is electron transfer which occurs when alkali metals or similar species are the initiators. Cationic polymerization, on the other hand, is initiated by electrophilic agents such as a protonic acid and a Lewis acid. Examples of Lewis acid initiators include AlCl₃, SnCl₄, BF₃, TiCl₄, AgClO₄, and I₂ in combination with a co-initiator such as H₂O or an organic halogen compound.

Although most of the ionic living polymerization techniques are not tolerant towards primary and secondary amino functional groups in the monomers to be polymerized, anionic polymerization of t-butylaminoethyl methacrylate is possible because of its relatively low basicity. The antimicrobial tBAEMA polymers can be prepared by anionic polymerization method described in “Living anionic homo- and block copolymerization of 2-(tert-butylamino)ethyl methacrylate” by Serge Creutz, Philippe Teyssie and Robert Jerome, J. Polymer Science (part A), vol 35 (10), 1997, 2035-2040 using a monomer to initiator molar ratio of from 5 to 100. Preferred initiators are diphenylmethyllithium with lithium chloride.

Typical controlled radical polymerization is provided by recent methods such as atom transfer radical polymerization (ATRP), nitroxide-mediated radical polymerization (NMP), reversible addition-fragmentation chain transfer polymerization (RAFT) and other related processes involving a degenerative transfer, such as macromolecular design via interchange of xanthates (hereinafter referred as MADIX).

ATRP is the most typical polymerization method to make antimicrobial polymers of low polydispersity.

ATRP is normally initiated by the redox reaction between an initiator comprising a transferable atom or group and catalyst comprising a transition metal complex in a lower oxidation state. The transferable atom or group (G) can be homolytically cleaved from the initiator by the catalyst, thereby oxidizing the catalyst to a high oxidation state and forming a radical thereby activating the initiator residual (A) for monomer addition. After the initiation, the ATRP process is mediated by the catalyst in a fast dynamic equilibrium between activating and deactivating the polymer chains via a similar homolytic atom or group transfer through the redox reaction.

Any transition metal complex capable of maintaining the dynamic equilibrium with the polymer chain may be used as the redox catalyst in ATRP. Suitable catalysts may be transition metal complexes of copper, ruthenium, iron, rhodium, nickel and palladium, molybdenum, and osmium. Preferred transition metal catalysts are copper complexes such as copper (I) halides with a ligand. The metal catalyst can be reduced form (e.g., Cu+), in oxide form (e.g., Cu+2), in atom form (e.g. Cu(0)) or mixture of all the metal forms in different valence. A particular ATRP process called “single electron transfer” (SET) living radical polymerization (LRP) uses only metal copper (Cu(0)) as initial catalyst, but the other valence forms of copper (Cu+ and Cu+2) are also generated in-situ and present during the polymerization process. In the so called reverse ATRP process, only metal in oxide form (e.g., Cu+2) is added initially, but metal in the reduced form (Cu+) is generated in-situ to make atom transfer radical polymerization work.

Suitable ligands for ATRP catalyst include but are not limit to bipyridine compounds, polydentate amines, terpyridyl and quadridentate amine bearing pyridine. Examples of pyridine compounds are 2,2′-bipyridine, 4,4′ substituted 2,2′-bipyridine (such as 4,4′-di(5-nonyl)-2,2′-bipyridine and 4,4′-diheptyl-2,2′-bipyridine), BIS(2-PYRIDINAL)ETHYLENEDIIMINE, tris-(2-pyridylmethyl)amine (TPMA). Examples of dentate amine ligands are hexa-N,N-substituted tris[2-(amino)ethyl]amine (TREN) such as tris[2-(N,N-dimethylamino)ethyl]amine (Me6TREN), 1,1,4,7,10,10-hexamethyltriethylenetetramine (HMTEA), and penta methyl diethylene triamine (Me₅DETA). Preferred ligands for ATRP catalyst are Me₆TREN, TPMA, and HMTEA. Tetradentated branched ligands such as Me6TREN and TPMA form highly active catalysts with copper halids such as CuBr and are the most suitable for the preparation of low MW antimicrobial tBAEMA oligomers at low temperature and low degrees of polymerization.

Suitable ATRP initiators include, but not limited to, halogenated alkanes, benzylic halides, α-haloesters, a-haloketones, alkyl and aryl sulfonyl chlorides. Preferred initiators are α-haloesters and α-haloketones, More preferred initiators are α-haloesters such as 2-haloisobutyrates and 2-halobutyrates. Examples of α-haloester initiators are ethyl 2-bromoisobutyrate (EBiB) and ethyl 2-bromobutyrate.

While not limiting the scope of the invention, it is believed that added antifungal activity against multicellular microorganism of the tBAEMA polymers may result when the tBAEMA polymer is of low molecular weight. Reduced molecular weight of the tBAEMA polymer may make the antimicrobial agent easier to penetrate and/or attach to multicellular structure of mold fungi for killing. See table 1 below.

The low molecular weight antimicrobial polymers formed from t-butylaminoethyl methacylate (tBAEMA) may be represented by formula (II)

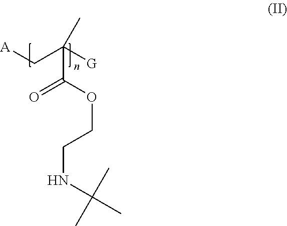

where n is from 2 to 100, and A and G are residual groups derived from initiator and chain transfer agent used in polymerization. Preferably n is from 5 to 60, and most preferably from 10 to 40.

A and G of formula (II) are residual groups derived from an initiator and optionally a chain transfer agent used in polymerization. The initiator may be selected from the group consisting of free radical polymerization initiators, atom transfer radical polymerization (ATRP) initiators, nitroxide-mediated radical polymerization (NMP) initiators, reversible addition-fragmentation chain transfer polymerization (RAFT) or macromolecular design via interchange of xanthates (MADIX), preferably atom transfer radical polymerization (ATRP), preferably the initiator is a free radical initiator selected from the group consisting of azo and peroxide initiators.

The mole percent of A+G ranges from about 1 to about 30, preferably about 1.5 to about 17, most preferably about 2.4 to about 9 mole percent based on the total moles of A+G and monomer units

If the process of polymerization for formula (II) is ATRP, A and G will be derived from alkyl halide initiator used for atom transfer radical polymerization (ATRP) polymerization. For example, A is an alkyl 2-isobutyrate radical and G is a halide which can be obtained by using an alkyl 2-haloisobutyrate ATRP initiator. Most especially, G is a bromide or an iodide, which may possibly contribute to enhance antifungal activity of the antimicrobial polymer of formula (I).

If the process is carried out via radical polymerization via azo or peroxide inititiators, A and G will be derived from azo and/or peroxide intitiators.

### Application of the Antimicrobial Polymer

The amount of the antimicrobial polymers effective as a wound dressing finish will vary form 0.001% to 20%, preferably from 0.001% to 10%, especially 0.001% to 5% by weight wherein the percent is based on the total weight of the wound dressing or preferably the gauze.

Gauze and wound dressing materials have a much higher surface area than most surfaces. For example the specific surface area weight of gauze fabric or dressing materials may range from about 20 g/m² to about 100 g/m², preferably 30 g/m² to 80 g/m² and especially 35 g/m² to 60 g/m². The solution concentration of 0.1 to 10% would then be translated into a 0.2 g/m² to 1000 g/m², 0.3 g/m² to 800 g/m², 0.35 g/m² to 60 g/m².

Thus, the antimicrobial wound dressing material may have a specific surface area weight ranging from at least 20 g/m², preferably at least 30 g/m² and especially at least 35 g/m². Specific surface area weight means the amount of loading in grams of the antimicrobial polymer per meter squared of wound dressing material.

Solvent and/or aqueous dispersions or solutions of the antimicrobial polymer of formula (I) may be used for applying the polymer onto the wound dressing or gauze.

Ethanol, methanol ethanol, methanol and isopropanol, ethers such as tetrahydrofunan (THF), and ketones such as methylethyl ketone (MEK) are good solvents for the antimicrobial polymer. Water and water/solvent mixtures are also possible.

Finishing of the wound dressing material, or especially gauze may be carried out by any means suitable for applying a coating or finish. Accordingly, the antimicrobial polymer may be applied by gravure coating, knife over roll coating, metering rod coating, immersion coating, dipping, spraying, spincoating, knife coating, curtain coating techniques, brush application, and reverse roll coating.

Dip coating can be realized by dipping or immersion of the gauze in the polymer solution, removing and drying. The treated gauze may contain a 0.001% to 20%, preferably 0.001 to 10%, most preferably 0.001 to 5.0%, especially 0.001 to 2 or 3 wt. % of the non-leachable antimicrobial polymer. The weight percent is based on the total weight of the untreated gauze. A gauze fabric can also be treated using various application methods typically used the textile industry.

Accordingly one of the preferred embodiments is an antimicrobial, non-leachable wound dressing which comprises a wound-dressing material, treated with a polymer formed from the monomer of formula (I)

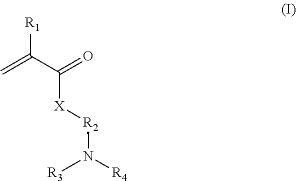

wherein R₁ is H or CH₃,
R₂ is C₁-C₅ alkyl bi-radical, preferably R₂ is C₂ bi-radical,
R₃ and R₄ are independently H or C₁-C₅ alkyl radical which can be linear or branched, preferably R₃ is hydrogen and R₄ is tert-butyl,
and X is a divalent radical of —O—, —NH— or —NR₅, preferably —NH, wherein R₅ is C₁-C₆ alkyl wherein the formed polymer is in the form of a coating or finish and the formed polymer ranges from about 0.01 to 20.0 wt. %, preferably 0.01 to about 10 wt. %, most preferably about 0.01 to about 5.0, especially 0.01 to 2, 3 or 3.5 wt. %, wherein the percent is based on the total weight of the untreated gauze

Furthermore the antimicrobial polymer is not only antimicrobial and antifungual but may be antiviral. Thus a method of protecting wound dressing from viral contamination is envisioned by incorporating or treating said wound dressing with an effective amount, preferably about 0.001 to 20.0 wt. %, most preferably 0.001 to about 10 wt. %, especially about 0.001 to about 5.0 and most especially 0.001 to 2, 3 or 3.5 wt. %, of the antimicrobial polymer formed from the monomer of formula (I) or the antimicrobial polymer formed from t-butylaminoethyl methacylate (tBAEMA) represented by formula (II) above wherein the wt. % is based on the total weight of the treated wound dressing.

An effective amount of antimicrobial for accomplishing an antimicrobial, antifungal or antiviral effect and also providing a non-leachable effect on the wound dressing means that the antimicrobial polymer may be applied or incorporated into the wound dressing in amounts as low as 2 ppm up to about 2, 3 or 3.5 wt. % wherein the wt. % and ppm are based on the total weight of the treated wound dressing, preferably the wound dressing is selected from the group consisting of fibers, nonwovens, sponges, absorptive packings, pads, cotton or cotton balls, band aids, sponges or tapes, especially a gauze.

The wound dressing material is preferably in the form of gauze, pads, band aids, absorptive packings, cotton or cotton balls, wound fillers, sponge or tapes, especially a gauze and the non-leachability is determined by ASTM E 2149 method using the specified pre-test and post-test designed for testing non-leachable antimicrobial surfaces.

It is also possible to treat the wound dressing materials, especially gauze with other antibacterial ingredients. Bactericides or bacteriostatic agents (component (d)), which influence the germ flora and kill off or inhibit the growth of bacteria, may also be present in the formulations. Many biocides are known for decades, such as silver, silver salts, triclosan, chlorohexidene, quaternary ammonium salts, polydimethyldiallylammonium chloride and polyhexamethylen-biguanid compounds. Typical examples are, in particular, phenoxyethanol. 5-Chloro-2-(2,4-dichlorophenoxy)-phenol, which is marketed by BASF SE Ludwigshaven, Germany under the name of Irgasan® (Triclosan) is particularly effective.

The following examples describe certain embodiments of this invention, but the invention is not limited thereto. It should be understood that numerous changes to the disclosed embodiments can be made in accordance with the disclosure herein without departing from the spirit or scope of the invention. These examples are therefore not meant to limit the scope of the invention. Rather, the scope of the invention is to be determined only by the appended claims and their equivalents.

### EXAMPLES

### Example 1

### Preparation of Low MW pTBAEMA with Narrow MW Distribution by the ATRP Process

Into a 50 mL three neck round bottom flask reactor are charged 0.1549 g (0.1 mMole) of CuBr, 0.075 g (0.02 mMole) of CuBr₂, 0.342 g of tris[2-(dimethylamino)ethyl]amine (Me₆TREN) and 5.50 g of dimethyl sulfoxide (DMSO). The reactor content is mixed and sparged with nitrogen for about 10 minutes. At the same time, 18.28 g (0.1 mole) of t-butylaminoethyl methacrylate (tBAEMA) and 1.93 g (0.01 mole) Ethyl 2-bromoisobutyrate (EBiB) are mixed and sparged with nitrogen in a drop funnel for 10 minutes. The reactant content in the drop funnel is added to the reactor under nitrogen sparging to start polymerization. After polymerization under nitrogen for about 2 hours, the reactor content is precipitated in 300 mL of hexane and stirred overnight. The residual catalysts are removed from bottom DMSO phase and the polymer is recovered from the hexane phase through rotary evaporation. The polymer is re-dissolved in 10 g of DMSO and precipitated in fresh boiling hexane again to further remove residual monomer and catalysts. The polymer was recovered again by rotary evaporation and then dried in a vacuum oven at 50° C. overnight. The purified polymer product is analyzed with gel permeation chromatography (GPC) to have a number average molecular weight (Mn) of 2,700 and a weight average molecular weight (Mw) of 4,400 using poly(methyl methacrylate) monodisperse molecular weight standards from Polymer Labs. The molecular weight polydispersity index (PDI=Mw/Mn) is 1.67.

### 1A

Preparation of tBAEMA Polymer

Following the procedure described in Example 1 of U.S. Pat. No. 6,096,800 using azobisisobutyronitrile (AIBN) initiator and tetrahydrofuran (THF) solvent, a tBAEMA homopolymer was prepared and characterized by GPC to have a weight average molecular weight (Mw) of 174,000 and a number average molecular weight of 63,000 (polydispersity index Mw/Mn=2.75).

### 1B

Preparation of tBAEMA Polymer

Following the same procedure of 1A except double the amount of the THF solvent to lower the initial monomer concentration, a lower MW tBAEMA homopolymer was prepared and characterized by GPC to have a weight average molecular weight (Mw) of 91,000 a number average molecular weight of 12,000 (polydispersity index Mw/Mn=7.40).

Examples 1, 1A and 1B were each compared for fungal activity against mold fungi A. niger.

Microbicidal activity is tested according to trivial modifications of the standard EN1040 test method. A bacterial suspension with a cell count of about 10⁷ cfu/m¹ is contacted with appropriate concentrations of the specific substances and the residual cell count is determined after contact time and incubation period. The resulting cell count reduction is compared to a water control.

Specifically, 1 g stock solution with an appropriate concentration of test products are mixed with 8 g water and then inoculated with 1 ml of the selected test organisms. After a given contact period, aliquots are taken, inactivated and diluted. The number of surviving bacteria per ml incubation assay is determined by plate count.

| Test organisms (inoculum) | type |
|---|---|
| Aspergillus niger ATCC 6275 | mould fungi |

Test concentration: 1000 ppm for A. niger fungi
Contact times: 5 and 30 minutes at 22° C.
Incubation: 24 h and 7 days at 30° C.

The test results are shown as log reduction of the initial count in Table 1. The data are expressed as measured microorganism concentration (cfu/mL) and log reduction compared to blank (H₂O reference). The tBAEMA sample of example 1 show additional microbial activity against mold fungi A. niger with a cell count reduction of 2 logs found after 30 min and 3 logs after 7 days whereas the two higher MW samples of examples 1A and 1B did not show any activity after 7 days.

TABLE 1
Test results of microcidal activity against fungi (molds) A. niger

| Sample (1000 ppm) | 30 min. | 1 h | 24 h | 7 days |
|---|---|---|---|---|
| Blank: cfu/mL | 2.90 × 10⁶ | 1.90 × 10⁶ | 2.00 × 10⁶ | 1.90 × 10⁶ |
| Example 1 | 2.50 × 10⁴ | 1.80 × 10⁴ | 1.50 × 10⁴ | 2.10 × 10³ |
| (Mw 4,400) cfu/mL | | | | |
| log reduction | 2.1 | 2.0 | 2.0 | 3.0 |
| Example 1A | 1.40 × 10⁶ | 1.20 × 10⁶ | 1.30 × 10⁶ | 1.80 × 10⁶ |
| (Mw 174,000) cfu/mL | | | | |
| log reduction | <1 | <1 | <1 | <1 |
| Example 1B | 2.30 × 10⁵ | 2.60 × 10⁵ | 2.30 × 10⁵ | 3.20 × 10³ |
| (Mw 91,000) cfu/mL | | | | |
| log reduction | 1.1 | <1 | <1 | 2.8 |

Comparative B did not show any activity until 7 days later while Example 1 showed activity (log 2 reduction) in less than 24 h. Thus the lower molecular material appears to be twice as effective as the higher molecular weight material at the same concentration.

### Example 2

### Preparation of High MW tBAEMA Homopolymer with Narrow Molecular Weight Distribution by Conventional Radical Polymerization Process

40 g of t-butylaminoethyl methacrylate (tBAEMA) monomer (M) and 160 g of tetrahydrofuran (THF) solvent are charged to a 500 mL reactor equipped with overhead condenser and agitator. The reactor content with overhead condenser is heated to 65° C. under agitation and nitrogen sparging for 1 hour. After 1 hour nitrogen sparging and the reaction temperature reaches 65° C., an initiator solution (I) comprising 0.4 g of AIBN (azobisisobutyronitrile) and 10 g of THF are added to the reactor slowly over about 60 minutes. The reactor is maintained at 70° C. under nitrogen blanket overnight. The reactor content is cooled down to room temperature. The final reaction product is added to 1 L of heptane under agitation. The polymer product is removed by filtration and wash with 300 mL of fresh heptane. The product is dried in a vacuum oven at 50° C. for 12 hours. The polymer product is analyzed with gel permeation chromatography (GPC) to have a number average molecular weight (Mn) of 54,000 g/mole and a weight average molecular weight (Mw) of 135,000 g/mole using poly(methyl methacrylate) monodisperse molecular weight standards from Polymer Labs. The molecular weight polydispersity index (PDI=Mw/Mn) is 2.62.

### Example 3

### Preparation of Low MW TBAEMA Homopolymer with Narrow Molecular Weight Distribution by Conventional Radical Polymerization Process

4800 g of tetrahydrofuran (THF) solvent is charged to a 10 L reactor equipped with overhead condenser and agitator. The reactor content with overhead condenser is heated to 65° C. under agitation and nitrogen sparging for 1 hour. After 1 hour nitrogen sparging and the reaction temperature reaches 65° C., 1200 g of t-butylaminoethyl methacrylate (tBAEMA) monomer (M) and an initiator solution (I) comprising 150 g of AIBN (azobisisobutyronitrile) and 1500 g of THF are added to the reactor slowly over about 180 minutes. The reactor is maintained at reflux temperature under nitrogen blanket and agitation during the M and I feeds and for additional 3 hours after the feeds. Monomer conversion is more than 95% after the polymerization reaction. The reactor content is heated to distill out about 5000 g of solvent. Fresh THF solvent (2000 g) is added to the reactor and distillation of solvent out of the reactor is repeated until residual monomer is less than 1%. The reactor content is cooled down to room temperature. The final solution polymer product contains 75% polymer solids. The polymer product is analyzed with gel permeation chromatography (GPC) to have a number average molecular weight (Mn) of 2,850 g/mole and a weight average molecular weight (Mw) of 6,900 g/mole using poly(methyl methacrylate) monodisperse molecular weight standards from Polymer Labs. The molecular weight polydispersity index (PDI=Mw/Mn) is 2.42.

### APPLICATION EXAMPLES

### Example 4

### Treatment of Medical Gauze with TBAEMA Hompopolymer

A TBAEMA homopolymer with Mw of 6,900 g/mole and PDI of 2.42 (polymer from Example 3) is used to prepare treatment solutions (A) with different polymer concentration in ethanol solution. The polymer concentration of the treatment solution is changed so as to adjust desired polymer loading level in the treated gauze. Gauze fabric made from a Rayon/polyester/cellulose blend (3″×3″ Hospital grade gauze pad by Johnson & Johnson) is immersed in a treatment solution (A). The treated fabric is taken out and excessive solution is removed simply by drip off (process D) or press squeeze (process P). The wet gauze fabric is weighed. The amount of the treatment solution absorbed by the fabric is determined by the weight increase of the wet fabric over the dry one and is used to calculate the polymer loading level based on the known polymer concentration of the treatment solution (A). The treated gauze fabric is dried in a fume hood at room temperature for 25 minute and then in an oven at 70° C. for 100 minutes.

TABLE 2
pTBAEMA treated J&J gauze samples (each gauze weighs
about 1.6 g). The exemplified gauze has a surface area
weight of approximately 40 g/m².

| Sample ID | Treatment | pTBAEMA loading |
|---|---|---|
| blank | Untreated | 0 |
| blank | ethanol only | 0 |
| 1 | D process | 20% |
| 2 | D process | 6.0% |
| 3 | D process | 3.5% |
| 4 | D process | 2.0% |
| 5 | D process | 1.0% |
| 6 | D process | 0.5% |
| 7 | P process | 10% |
| 8 | P process | 3.0% |
| 9 | P process | 2.0% |
| 10 | P process | 1.0% |
| 11 | P process | 0.5% |
| 12 | P process | 0.3% |

### Example 5

### Antimicrobial Test for Non-Leachable Antimicrobial Gauze

The gauzes prepared in Example 4 are tested using modified ASTM E 2149-2001, a standard method to determine antimicrobial activity and non-leachability.

The products (in this case treated gauze) are first analyzed for leaching antimicrobial agent (pre-test) using a standard assay (CG 147 agar diffusion). No leaching of antimicrobial is evident if no zone of inhibition (ZOI) is observed. The gauze fabric is shaken in a microbial culture containing known concentration (cfu/ml) of test microorganism in a jar for certain amount time. Concentrations (cfu/ml) of microorganisms in the jar containing the antimicrobial product are determined and compared to either the jar containing only microbial suspension or the jar containing the control objects (e.g., blanks). A product is said to be “antimicrobial” if it produces a substantial reduction (log reduction>3) relative to either the inoculum or object controls.

A post test to further confirm non-leachability is also required after the antimicrobial activity testing. The post test consists of shaking the antimicrobial sample in the culture medium the same way as the ASTM 2149 test but without the test microorganism. Aliquots of the culture medium solution after shaking is tested in an agar cup test to check whether effective amounts of antimicrobial compound is leached out during the 2149 test. Presence of ZOI in the agar cup test indicates leaching.

Results of antimicrobial bioactivity test of by ASTM E2149 method are shown in Tables 3 to 6.
Sample preparation: ASTM E 2149: gauzes are tested on weight base:
0.5 g/25 ml or 0.25 g/12.5 ml
Agar diffusion Test (CG 147): gauzes were cut into pieces of 2 cm diameter
Remarks: two layers of the gauzes were used in all test systems
Test methods: CG 204e/ASTM 2149-01
   CG 147e/agar diffusion test
   ASTM 2149-01 12 Procedure for determining presence of
   antimicrobial leaching (“Post-test”)
Test organism: Staphylococcus aureus DSM 799 (˜) ATCC 6538
   Escherichia coli DSM 682 (˜) ATCC 10536

TABLE 3
Test results of antimicrobial bioactivity by ASTM E2149 method
Method: modified ASTM E 2149-2001

| | | | | | E. coli | S. aureus |
|---|---|---|---|---|---|---|
| | | | E. coli | S. aureus | [log- | [log- |
| Sample | Formulation | Aspect | [cfu/mL] | [cfu/mL] | reduction] | reduction] |
| | growth control 0 h | | 2.6E+05 | 3.5E+05 | | |
| | growth control 24 h | | 5.8E+05 | 4.6E+05 | | |
| Blank | blank untreated | 0 h | 2.9E+05 | 3.8E+05 | | |
| | J&J gauze fabrics | 24 h | 6.5E+05 | 4.1E+05 | 7.7E+05 | 4.3E+05 |
| | | | 8.8E+05 | 4.5E+05 | | |
| Blank | Ethanol solution treated | | 5.8E+05 | 6.5E+05 | <1 | <1 |
| | only | | 5.9E+05 | 5.7E+05 | <1 | <1 |
| 1 | D process, pTBAEMA | | <1 | <1 | >5.9 | >5.6 |
| | loading 20% | | <1 | <1 | >5.9 | >5.6 |
| 2 | D process, pTBAEMA | | <1 | <1 | >5.9 | >5.6 |
| | loading 6.0% | | <1 | <1 | >5.9 | >5.6 |
| 3 | D process, pTBAEMA | | <1 | <1 | >5.9 | >5.6 |
| | loading 3.5% | | <1 | <1 | >5.9 | >5.6 |
| 4 | D process, pTBAEMA | | <1 | <1 | >5.9 | >5.6 |
| | loading 2.0% | | <1 | <1 | >5.9 | >5.6 |
| 5 | D process, pTBAEMA | | <1 | <1 | >5.9 | >5.6 |
| | loading 1.0% | | <1 | <1 | >5.9 | >5.6 |
| 6 | D process, pTBAEMA | | <1 | <1 | >5.9 | >5.6 |
| | loading 0.5% | | <1 | <1 | >5.9 | >5.6 |
| 7 | P process, pTBAEMA | | <1 | <1 | >5.9 | >5.6 |
| | loading 10% | | <1 | <1 | >5.9 | >5.6 |
| 8 | P process, pTBAEMA | | <1 | <1 | >5.9 | >5.6 |
| | loading 3.0% | | <1 | <1 | >5.9 | >5.6 |
| 9 | P process, pTBAEMA | | <1 | <1 | >5.9 | >5.6 |
| | loading 2.0% | | <1 | <1 | >5.9 | >5.6 |
| 10 | P process, pTBAEMA | | <1 | <1 | >5.9 | >5.6 |
| | loading 1.0% | | <1 | <1 | >5.9 | >5.6 |
| 11 | P process, pTBAEMA | | <1 | <1 | >5.9 | >5.6 |
| | loading 0.5% | | <1 | <1 | >5.9 | >5.6 |
| 12 | P process, pTBAEMA | | <1 | <1 | >5.9 | >5.6 |
| | loading 0.3% | | <1 | <1 | >5.9 | >5.6 |
| Negative | LSR MED 4850 | | 6.5E+05 | 1.3E+05 | | |
| reference | | | | | | |
| Positive | Vygon catheter | | <1 | <1 | | |
| reference | | | | | | |
| Adjust cell | | | 1.9E+09 | 4.5E+08 | | |
| number | | | | | | |

TABLE 4
Test results of antimicrobial bioactivity by ASTM E2149
method for gauze treated with low molecular weight (LMW)
(prepared according to example 3) and high molecular weight
(HMW) pTBAEMA (prepared according to example 2)

| | E. coli | S. aureus |
|---|---|---|
| pTBAEMA loading | Log reduction | Log reduction |
| Blank gauze | −0.05 | 1.04 |
| 0.04% LMW (Mw = 7k) | 5.73 | 6.15 |
| 0.02% LMW (Mw = 7k) | 5.73 | 6.15 |
| 0.08% HMW (Mw = 135k) | 5.73 | 6.15 |
| 0.04% HMW (Mw = 135k) | 4.42 | 4.66 |
| 0.02% HMW (Mw = 135k) | 4.37 | 6.15 |

Clearly the low molecular weight pTBAEMA is more effective at lower concentrations than the higher molecular weight pTBAEMA.

TABLE 5
Agar diffusion test for leachability

| | | Staphylococcus | | Escherichia | |
|---|---|---|---|---|---|
| | | aureus | | coli | |
| | | ATCC | | ATCC | |
| | | 6538 | | 10536 | |
| | | ZI | VR | ZI | VR |
| B | blank untreated J&J gauze fabrics | 0 | 0 | 0 | 0 |
| B | Ethanol solution treated only | 0 | 0 | 0 | 0 |
| 1 | D process, pTBAEMA loading 20% | 1 | 4 | 1 | 4 |
| 2 | D process, pTBAEMA loading 6.0% | 1 | 4 | 2 | 4 |
| 3 | D process, pTBAEMA loading 3.5% | 0 | 0 | 0 | 0 |
| 4 | D process, pTBAEMA loading 2.0% | 0 | 0 | 0 | 0 |
| 5 | D process, pTBAEMA loading 1.0% | 0 | 0 | 0 | 0 |
| 6 | D process, pTBAEMA loading 0.5% | 0 | 0 | 0 | 0 |
| 7 | P process, pTBAEMA loading 10% | 2 | 4 | 3 | 4 |
| 8 | P process, pTBAEMA loading 3.0% | 0 | 0 | 1 | 4 |
| 9 | P process, pTBAEMA loading 2.0% | 0 | 0 | 0 | 0 |
| 10 | P process, pTBAEMA loading 1.0% | 0 | 0 | 0 | 0 |
| 11 | P process, pTBAEMA loading 0.5% | 0 | 0 | 0 | 0 |
| 12 | P process, pTBAEMA loading 0.3% | 0 | 0 | 0 | 0 |

All tests were performed twice and both results are given in the table.
Legend: ZI = zone of inhibition in mm
4 = no growth (good activity)
VR = Vinson Rating, for growth under the disc
2 = single colonies under the disc (moderate activity)
0 = strong growth (no activity)
(L. J. Cinson et al. J Pharm. Sci. 50, 827-830, 1961)

TABLE 6
Post test (according to ASTM E 2149-01) for leachability

| | Inhibition zone |
|---|---|
| Untreated blank | 0/0 |
| All pTBAEMA treated gauzes | 0/0 |

Performance of the biocide activity of the antimicrobial polymer is excellent as more than 5 log reduction in bacteria concentration is achieved with pTBAEMA treated gauze even at loading level as low as 0.3% (Table 1). No ZOI is observed in the agar diffusion pre-test for loading level below 3% (table 2). No ZOI is observed in the post test for all loading levels up to 20% (Table 3). The antimicrobial polymer is thus qualified as a non-leaching antimicrobial agent by ASTM E 2149 method.

1. An antimicrobial, non-leachable wound dressing which comprises a wound-dressing material, treated with a polymer formed from a monomer of formula (I) 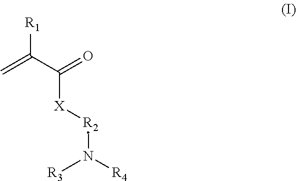
wherein R₁ is H or CH₃,
R₂ is C₁-C₅ alkyl bi-radical, preferably R₂ is C₂ bi-radical,
R₃ and R₄ are independently H or C₁-C₅ alkyl radical which can be linear or branched,
preferably R₃ is hydrogen and R₄ is tert-butyl,
and X is a divalent radical of —O—, —NH— or —NR₅, preferably —NH, wherein R₅ is C₁-C₆ alkyl.

2. The antimicrobial, non-leachable wound dressing material according to claim 1, wherein the non-leachability is determined by ASTM E 2149 method using the specified pre-test and post-test designed for testing non-leachable antimicrobial surfaces.

3. The antimicrobial, non-leachable wound dressing material according to claim 1, wherein the formed polymer is in the form of a coating or finish and the antimicrobial polymer ranges from about 0.001 to 5.0 wt. %, wherein the percent is based on the total weight of the untreated wound dressing material.

4. The antimicrobial wound dressing material according to claim 1, wherein the wound-dressing material is in the form of gauze, pads, band aids, absorptive packings, cotton or cotton balls, wound fillers, sponges or tapes, especially a gauze.

5. The antimicrobial wound dressing material according to claim 4, wherein the gauze, pads, band aids, absorptive packings, cotton or cotton balls, wound fillers and tapes are formed from materials
selected from the group consisting of synthetic materials, blends of synthetic materials and blends of synthetic materials with cotton, cellulose or cellulose derivatives.

6. The antimicrobial wound dressing material according to claim 5, wherein the gauze, pads, band aids, absorptive packings, cotton or cotton balls, sponges, wound fillers or tapes are at least partially formed from synthetic materials.

7. The antimicrobial wound dressing material according to claim 6, wherein the synthetic materials are at least partially rayon, polyester, polyethylene, polypropylene and blends thereof with cellulose or cellulose derivative.

8. The antimicrobial wound dressing material according to claim 4 wherein the dressing material has a specific surface area weight ranging from at least 20 g/m².

9. The antimicrobial wound dressing material according to claim 1, wherein the monomer of formula (I) is selected from the group consisting of tert-butylaminoethyl(meth)acrylate (tBAEMA), 2-dimethylaminoethyl(meth)acrylate, 2-diethylaminoethyl(meth)acrylate, 3-dimethylaminopropyl(meth)acrylate, N-3-dimethylaminopropyl(meth)acrylamide, and N-3-diethylaminopropyl(meth)acrylamide.

10. The antimicrobial wound dressing according to claim 1, wherein the formed polymer has a linear, random, block, brush, comb, grafted, hyperbranched or star architecture.

11. The antimicrobial wound dressing according to claim 1, wherein the weight average molecular weight of the polymer formed from the monomer of formula (I) ranges from 400 to 20,000 g/mole.

12. The antimicrobial wound dressing according to claim 1, which further contains medicinal ingredients or additional antimicrobial ingredients wherein the additional antimicrobial ingredients are selected from the group consisting of silver, silver salts, quaternary ammonium salts, polydimethyldiallylammonium chloride, chlorohexidine, polyhexamethylen-biguanid compounds and phenoxyethanol.5-Chloro-2-(2,4-dichlorophenoxy)-phenol (Triclosan).

13. A method of forming a antimicrobial non-leachable wound dressing material comprising the step
a) treating a wound dressing material with a polymer formed from a monomer of formula (I) 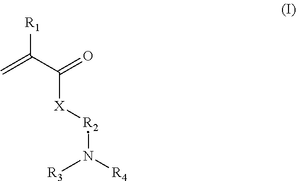
wherein R₁ is H or CH₃,
R₂ is C₁-C₅ alkyl bi-radical, preferably R₂ is C₂ alkyl bi-radical,
R₃ and R₄ are independently H or C₁-C₅ alkyl radical, which can be linear or branched,
preferably R₃ is hydrogen and R₄ is tert-butyl, and X is a divalent radical of —O—, —NH— or —NR₅, preferably —NH, wherein R₅ is C₁-C₆ alkyl
wherein the formed polymer is optionally dissolved or dispersed in a liquid.

14. The method according to claim 13, wherein the non-leachability is determined by ASTM E 2149 method using the specified pre-test and post-test designed for testing non-leachable antimicrobial surfaces.

15. The method according to claim 13, wherein the formed polymer is in the form of a coating or finish and the antimicrobial polymer ranges from about 0.001 to about 5.0 wt %, wherein the percent is based on the total weight of the untreated wound dressing material.

16. The method according to claim 13 wherein the wound dressing material is in the form of a gauze, pads, band aids, absorptive packings, cotton or cotton balls, wound fillers, sponges and tapes (medical).

17. The method according to claim 13, wherein the gauze, pads, band aids, absorptive packings, cotton or cotton balls, wound fillers, sponges and tapes are formed from materials selected from the group consisting of synthetic materials, blends of synthetic materials and blends of synthetic materials with cotton, cellulose or cellulose derivatives.

18. The method according to claim 16, wherein the material is at least partially formed from a synthetic material.

19. The method according to claim 13 wherein the weight average molecular weight of the polymer formed from the monomer of formula (I) ranges from about 400 to 20,000 g/mole.

20. A method of protecting a wound dressing from viral contamination by incorporating or treating said wound dressing with an effective amount the antimicrobial polymer according to claim 1.
